(19) United States
(12) Patent Application Publication
(10) Pub. No.: US 2006/0100665 A1
(43) Pub. Date: May 11, 2006

(54) Apparatus for sealing punctures in blood vessels

(75) Inventors: Randolf Von Oepen, Los Altos Hills, CA (US); Gerd Seibold, Ammerbuch (DE); Ib Erling Joergensen, Haigerloch (DE); Kenneth J. Michlitsch, Livermore, CA (US); Stevan Nielsen, Rottenberg (DE); Tommy Conzelmann, Rangendingen (DE); Bodo Quint, Rottenberg (DE)

Correspondence Address: LUCE, FORWARD, HAMILTON & SCRIPPS LLP, 11988 EL CAMINO REAL, SUITE 200, SAN DIEGO, CA, 92130, US

(73) Assignee: ABBOTT LABORATORIES VASCULAR ENTERPRISES, LIMITED, DUBLIN (IE)

(21) Appl. No.: 10/523,217

(22) PCT Filed: Jul. 25, 2003
(86) PCT No.: PCT/EP03/08245

§ 371 Date: Oct. 19, 2005

Related U.S. Application Data
(60) Provisional application No. 60/401,222, filed on Aug. 1, 2002.

(51) Int. Cl. A61B 17/04

(52) U.S. Cl. 606/232

(57) ABSTRACT

Apparatus is provided for sealing a puncture in a blood vessel using at least one device that applies an internal compressive force upon a distal surface of the vessel. The devices preferably are deployable distal of the distal surface of the vessel, then retracted proximally to provide the internal compressive force, e.g., to promote coagulation and facilitate sealing of the puncture. Additionally, the devices of the present invention optionally may be used in combination with known techniques that provide an external compressive force upon an exterior surface of a patient's skin to expedite sealing of the puncture.

### FIELD OF THE INVENTION

The present invention relates to apparatus for sealing punctures in blood vessels. More specifically, the invention relates to one or more devices that may be deployed in tissue distal of a vessel having a puncture to apply an internal compressive force upon the vessel to facilitate sealing of the puncture.

### BACKGROUND OF THE INVENTION

A large number of medical diagnostic and therapeutic procedures involve the percutaneous introduction of instrumentation into the blood vessel. For example, coronary angioplasty, angiography, atherectomy, stenting, and numerous other procedures often involve accessing the vasculature through placement of a catheter or other device in a patient's femoral artery or other blood vessel. Once the procedure is completed and the catheter or other diagnostic or therapeutic device is removed, bleeding from the resultant vascular puncture must be stopped.

Traditionally, a medical practitioner applies external pressure to the puncture site to stem bleeding until hemostasis occurs (i.e., when the clotting and tissue rebuilding have sealed the puncture). This method, however, presents numerous problems. In some instances, this pressure must be applied for up to an hour or more, during which time the patient is uncomfortably immobilized. In addition, there exists a risk of hematoma since bleeding from the puncture may continue until sufficient clotting occurs, particularly if the patient moves during the clotting process. Furthermore, application of external pressure to stop bleeding may be unsuitable for patients with substantial amounts of subcutaneous adipose tissue since the skin surface may be a considerable distance from the puncture site, thereby rendering external compression less effective.

Another traditional approach to subcutaneous puncture closure comprises having a medical practitioner internally suture the vessel puncture. This method, however, often requires a complex procedure and requires considerable skill by the medical practitioner.

Apparatus and methods also are known in which a plug is introduced into the vessel puncture to cover the puncture and promote hemostasis. One example of such a plug is described in U.S. Pat. No. 5,061,274 to Kensey and comprises a plug made from animal-derived collagen. Such apparatus may be unsuitable for some patients due to an adverse immunological reaction to animal-derived collagen. Furthermore, a plug inserted into the puncture tract may be dislodged into the vessel during the healing process due to the application of pressure to the wound, potentially causing stenosis of the vessel.

In view of these drawbacks of previously known devices, it would be desirable to provide apparatus for sealing a puncture within a vessel that provides an internal compressive force upon the vessel.

It also would be desirable to provide apparatus for sealing a puncture within a vessel that may provide the internal compressive force in conjunction with an external compressive force applied to an exterior surface of a patient's skin.

It further would be desirable to provide apparatus for sealing a puncture within a vessel that does not require a physician to manually maintain the compressive forces imposed.

It still further would be desirable to provide apparatus for sealing a puncture within a vessel that is biodegradable.

### SUMMARY OF THE INVENTION

In view of the foregoing, it is an object of the present invention to provide apparatus for sealing a puncture within a vessel that provides an internal compressive force upon the vessel.

It also is an object of the present invention to provide apparatus for sealing a puncture within a vessel that may provide the internal compressive force in conjunction with an external compressive force applied to an exterior surface of a patient's skin.

It further is an object of the present invention to provide apparatus for sealing a puncture within a vessel that does not require a physician to manually maintain the compressive forces imposed.

It further is an object of the present invention to provide apparatus for sealing a puncture within a vessel that is biodegradable.

These and other objects of the present invention are accomplished by providing apparatus for sealing a puncture within a vessel that comprises at least one device that may be deployed in tissue distal of the vessel to apply an internal compressive force upon the vessel. In the context of the present invention, the term “internal compressive force” refers generally to a force imposed at a location distal of a vessel and occurring in proximal direction toward a skin puncture. By contrast, an “external compressive force” is defined herein as a force imposed at a location near a skin puncture and occurring in a distal direction toward the vessel.

In a first embodiment of the present invention, the device preferably comprises a bar having a bore and a filament disposed through the bore. The bar and the filament are provided in a contracted state within a delivery sheath, for example, a hypodermic needle, having proximal and distal ends and a sharpened tip at the distal end. The tip of the delivery sheath is advanced distally to pass through the original puncture, which had previously been formed in a proximal lateral surface of the vessel. The tip further is advanced to pierce through a distal lateral surface of the vessel and is positioned distal of the vessel.

The bar then is ejected from within the confines of the delivery sheath, e.g., by advancing a push rod, to cause the bar to deploy distal of the vessel. First and second ends of the filament then may be retracted proximally from outside of a patient's body to retract the bar against the distal lateral surface of the vessel and apply an internal compressive force thereto. The internal compressive force causes coagulation within the punctures that facilitates sealing of the proximal and distal punctures.

After the punctures are effectively sealed, the first end of the filament may be retracted to cause the second end to be pulled through the bore of the bar and from within the patient's body. The bar, which preferably is biodegradable, remains in the patient's body after the filament is removed.

To expedite sealing of the puncture, the first and second ends of the filament may be tensioned within a tensioning device disposed on an exterior surface of a patient's skin. The use of the tensioning device advantageously permits a physician to retract the first and second ends of the filament to apply the internal compressive force via the bar, then lock the filament within the tensioning device so that the physician need not manually retain the compressive force upon the vessel. Additionally, the tensioning device serves to apply an external compressive force to the skin to enhance compression of the vessel.

In an alternative embodiment of the present invention, apparatus comprising at least one wire configured to self-deploy to a predetermined shape is provided in a contracted state confined within a body having at least one hollow needle. The wire includes a distal end having a sharpened tip configured to pierce through tissue surrounding the vessel. Preferably, the wire is configured to self-deploy to a predetermined shape, for example, a hook shape, in a deployed state.

In operation, the one or more hollow needles pierce through tissue surrounding a vessel having a puncture with the wires provided in the contracted state. The body is advanced to dispose the hollow needles at a location distal of the vessel, and the one or more wires then are advanced to cause the tip of the wires to pierce through tissue distal of the vessel. The wires self-deploy to the predetermined shape, e.g., the hook shape, distal of the vessel as they are advanced distal of the hollow needles.

With the wires in the deployed state, the wires are retracted proximally to cause the deployed hook to apply an internal compressive force upon the vessel. The internal compressive force causes coagulation at the puncture site that facilitates sealing of the puncture. Additionally, an external compressive force may be applied when the distal end of the body is pushed against a patient's skin. In a preferred embodiment, a first wire and first hollow needle are used in conjunction with a second wire and second hollow needle so that compressive forces are applied at two opposing locations about the vessel.

In another alternative embodiment of the present invention, apparatus comprising at least one wire configured to self-deploy to a predetermined shape is provided in a contracted state within a delivery sheath. The one or more wires each include a distal end having a sharpened tip configured to pierce through tissue surrounding the vessel. Preferably, the wire is configured to self-deploy to a predetermined arcuate shape, for example, a semicircular or circular shape, in a deployed state.

In operation, the delivery sheath is placed in the puncture tract leading to the vascular puncture site. The delivery sheath is positioned and/or configured such that the one or more wires may exit the delivery sheath in proximity to the vessel without entering the lumen of the vessel. For example, the delivery sheath may be positioned in the puncture tract such that the wires may exit a distal end of the sheath and be positioned in the puncture tract proximal of the puncture site. Alternatively, the sheath may comprise one or more side ports through which the one or more wires may exit the sheath proximal of the puncture site.

As the wires exit the sheath, they resume their predetermined, preferably arcuate shape. The one or more wires circumferentially pierce through tissue surrounding the vessel until their distal ends have at least crossed an imaginary plane on the distal side of the vessel, the imaginary plane including a diameter of the vessel passing from the puncture site through the vessel's midpoint to the distal side of the vessel.

Once positioned in the deployed state across the imaginary plane, the wires are proximally retracted to cause the arcuate shape to apply an internal compressive force upon the vessel. The internal compressive force causes coagulation at the puncture site that facilitates sealing of the puncture. To expedite sealing of the puncture, the proximal ends of the one or more wires may be tensioned within the previously described tensioning device disposed on an exterior surface of a patient's skin. The proximal ends of the wires may be locked within the tensioning device so that the physician need not manually retain compression upon the vessel. Additionally, the tensioning device may serve to apply an external compressive force to the skin to enhance compression of the vessel.

In a preferred embodiment, only a single wire having a predetermined shape is provided. The wire preferably has at least a semicircular shape, and may have a full circular shape that fully encircles the vessel. The radius of curvature of the predetermined shape preferably is greater than the radius of curvature of the vessel.

In the full circular embodiment, the distal end of the wire optionally may be recaptured within the delivery sheath, or may be recaptured using other capture apparatus disposed within the puncture tract. With the distal end of the wire recaptured, the sheath may be retracted proximally to apply the internal compressive force. Additionally, the proximal and/or distal ends of the wire may be locked within the tensioning device to provide the external compressive force.

In an alternative embodiment, two wires having predetermined shapes are provided. The wires cross the imaginary plane from opposite sides such that they overlap distal of the vessel, and compressive forces are distributed between the two wires, thereby reducing a risk of vessel dissection. As will be apparent to those of skill in the art, any number of wires having predetermined shapes may be provided.

### BRIEF DESCRIPTION OF THE DRAWINGS

Further features of the invention, its nature and various advantages will be more apparent from the accompanying drawings and the following detailed description of the preferred embodiments, in which:

FIGS. 1A-1D are, respectively, a side view of a first puncture sealing device, side views of alternative puncture sealing devices, and a perspective view of an alternative puncture sealing device provided in accordance with a first embodiment of the present invention;

FIGS. 2A-2B are, respectively, a side view and a side sectional view of apparatus for delivering the puncture sealing device of FIG. 1A;

FIGS. 3A-3F are side sectional views illustrating an exemplary method of using the apparatus of FIG. 2;

FIGS. 4A-4C are, respectively, a side view and side sectional views of an alternative puncture sealing device of the present invention in contracted and deployed states;

FIGS. 5A-5E are, respectively, a perspective view and a top view illustrating a preferred technique for deploying the apparatus of FIG. 4, and perspective views illustrating an exemplary method of using the apparatus of FIG. 4;

FIGS. 6A-6C are side sectional views illustrating an alternative embodiment of the present invention in contracted and deployed states;

FIG. 7 is a side sectional view illustrating an alternative embodiment of the device of FIGS. 6A-6C; and

FIGS. 8A-8D are perspective views illustrating an exemplary method of using the apparatus of FIG. 6.

### DETAILED DESCRIPTION OF THE INVENTION

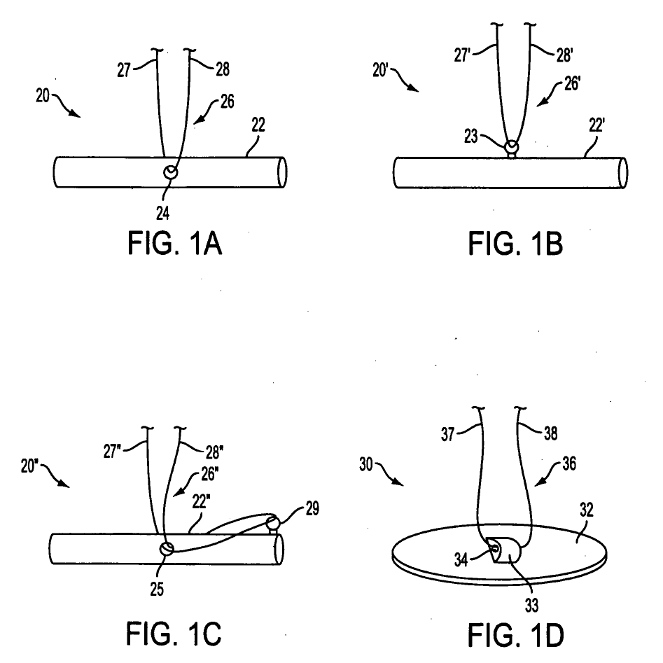

Referring now to FIG. 1A, a first embodiment of a puncture sealing device constructed in accordance with principles of the present invention is described. In FIG. 1A, puncture sealing device 20 comprises cylindrically-shaped bar 22 having proximal and distal ends and bore 24 extending laterally through a central region of bar 22. Bar 22 alternatively may comprise a rectangular or any other cross-section. Filament 26, having first end 27 and second end 28, extends through bore 24 and may be used to manipulate the positioning of bar 22, as described hereinbelow. Preferably, bar 22 comprises a biocompatible plastic or metal alloy, or a biodegradable material such as polyglycolic acid. Filament 26 may comprise a biocompatible wire, or more preferably a conventional suture material, for example, a biodegradable suture material.

In FIG. 1B, alternative puncture sealing device 20′ preferably is provided in accordance with bar 22 of FIG. 1A with the exception that bar 22′ preferably is solid and comprises first eyelet 23 coupled to a central region of bar 22′, e.g., using a solder or weld. Filament 26′ extends through first eyelet 23 and may be used to manipulate the positioning of bar 22′, as described hereinbelow.

Referring to FIG. 1C, alternative puncture sealing device 20″ preferably is provided in accordance with bar 22 of FIG. 1A with the exception that bar 22″ comprises central bore 25 extending laterally through a central region of bar 22″ and further comprises end eyelet 29 coupled to the distal end of bar 22″. Filament 26″ preferably extends through central bore 25, then through end eyelet 29, and optionally also may extend back through central bore 25 after passing through eyelet 29, as shown in FIG. 1C. Alternatively, filament 26″ may just extend once through central bore 25 and then through end eyelet 29. By retracting either first end 27″ or second end 28″ of filament 26″, it is possible to facilitate horizontal positioning of bar 22″ within a patient's tissue for purposes described hereinbelow. As will be apparent to those of skill in the art, bar 22″ alternatively may be provided with a central eyelet and an end bore, with central and end eyelets, or with central and end bores.

In FIG. 1D, alternative puncture sealing device 30 is illustrated as comprising substantially flat member 32 having an oval-shaped configuration. Member 32 comprises bore 34 extending through protruding region 33, which preferably is provided in a central region of member 32, as shown in FIG. 1B. Filament 36 having first end 37 and second end 38 extends through bore 34 to perform the functions described hereinbelow. It will be apparent to those skilled in the art that while bars 22 and member 32 of FIGS. 1A-1D are illustratively shown having cylindrical and oval-shaped configurations, respectively, other configurations advantageously may be provided to perform the functions described hereinbelow.

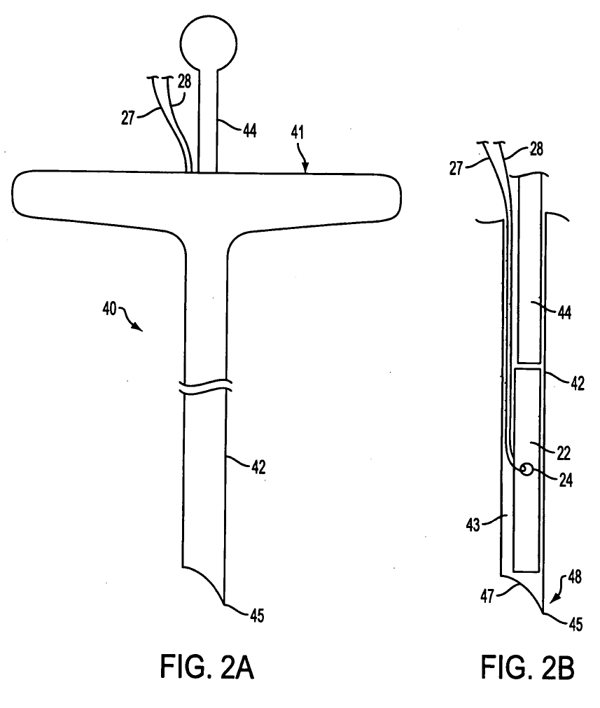

Referring now to FIG. 2, puncture sealing apparatus 40 of the first embodiment of the present invention preferably comprises delivery sheath 42 having proximal and distal ends and lumen 43 extending therebetween. The proximal end of delivery sheath 42 preferably comprises optional handle 41 that is configured to be grasped by a physician. Distal end 48 of delivery sheath 42 comprises sharpened tip 45 and opening 47, which is in fluid communication with lumen 43, as shown in FIG. 2B. Distal end 48 may comprise, for example, a standard hypodermic needle attached to handle 41. Distal end 48 preferably comprises a significantly smaller cross sectional area than optional handle 41 of delivery sheath 42. Bar 22 and filament 26 of FIG. 1A preferably are used in conjunction with delivery sheath 42, as described hereinbelow.

Apparatus 40 preferably further comprises push rod 44 having proximal and distal ends. Delivery sheath 42 is sized so that bar 22, push rod 44, and first and second ends 27 and 28 of filament 26 may be provided in a contracted state within lumen 43. In the contracted state, the distal end of push rod 44 is disposed just proximal of bar 22.

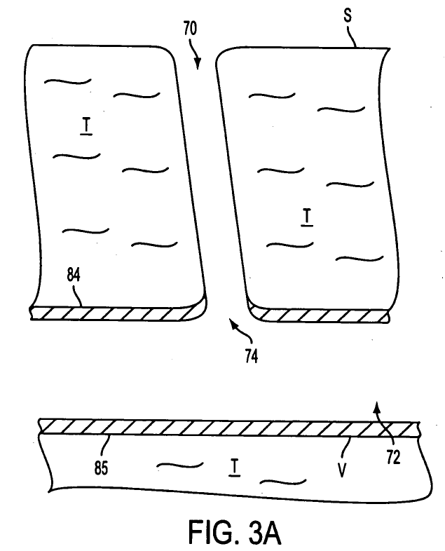

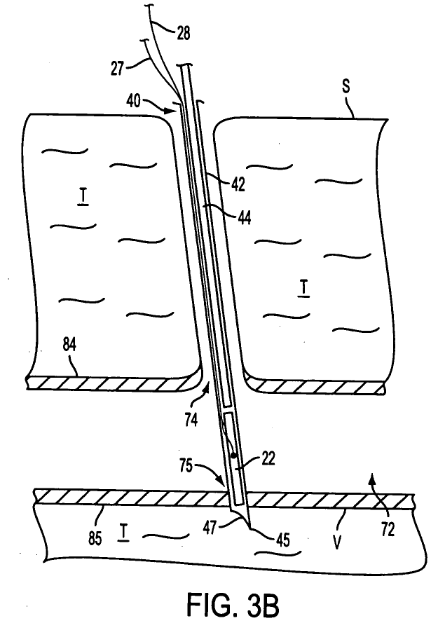

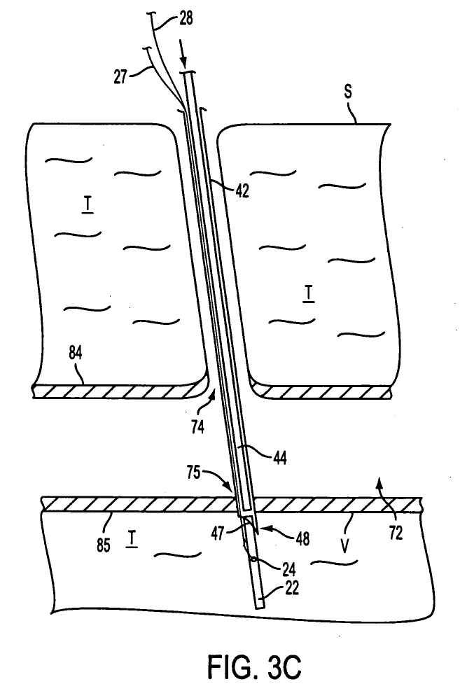

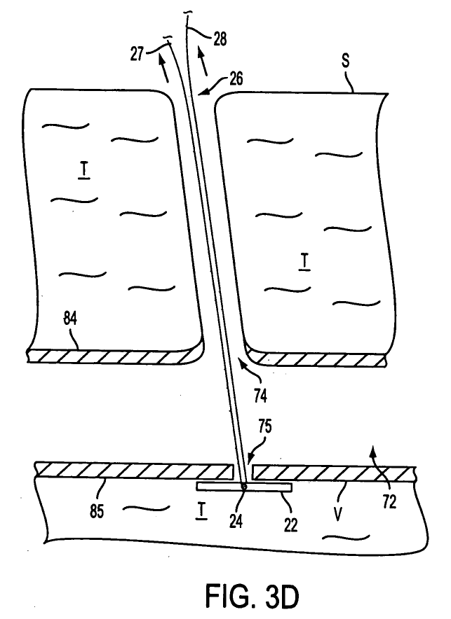

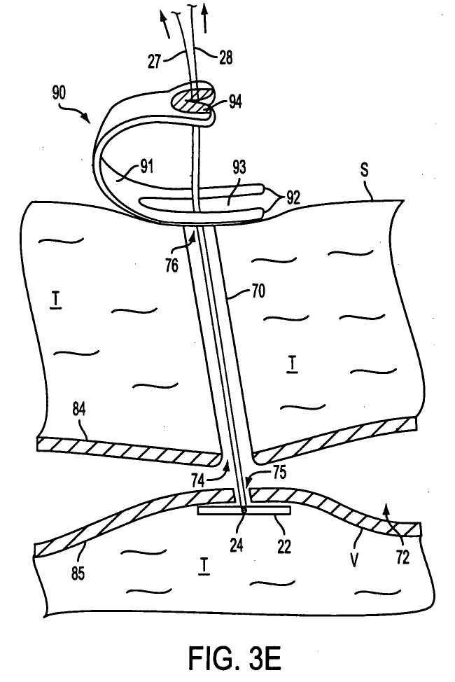

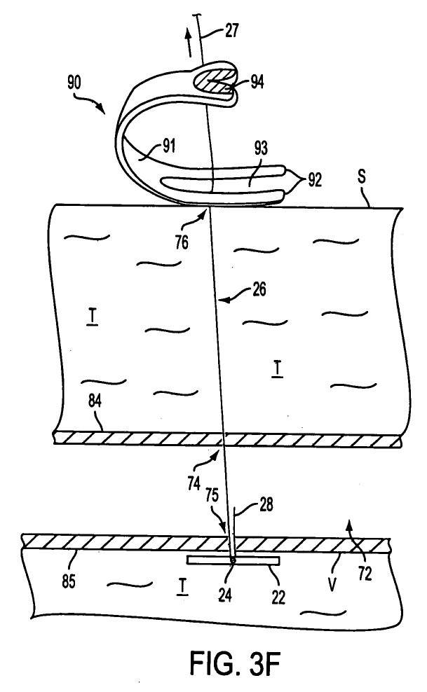

Referring now to FIG. 3, an exemplary method of using puncture sealing apparatus 40 of FIG. 2 to seal a puncture in a vessel is described. In FIG. 3A, proximal puncture 74 has been formed in proximal lateral surface 84 of vessel V, which is situated within tissue T. Puncture tract 70 has been formed and permits fluid communication between an exterior surface of a patient's body and lumen 72 of vessel V. Puncture tract 70 may have been formed, for example, as a means for introducing a guidewire and/or catheter into vessel V to perform a variety of medical procedures.

Referring now to FIG. 3B, a first step for using puncture sealing apparatus 40 of FIG. 2 in accordance with principles of the present invention is described. Sharpened tip 45 at the distal end of delivery sheath 42 is inserted into a patient's body, preferably via preexisting puncture tract 70 of FIG. 3A. Specifically, sharpened tip 45 is inserted through skin S, tissue T, proximal puncture 74, and through lumen 72 of vessel V. To ensure that sharpened tip 45 of apparatus 40 does not accidentally pierce tissue T during delivery, apparatus 40 optionally may be delivered through an external sheath, such as an introducer or guiding catheter.

Sharpened tip 45 further is advanced distally through lumen 72 to pierce through a distal wall of vessel V, such that sharpened tip 45 is again disposed within tissue T distal of vessel V, as shown in FIG. 3B. The piercing of the distal wall of vessel V forms distal puncture 75 in distal lateral surface 85 of vessel V, which is substantially diametrically opposing proximal puncture 74 in proximal lateral surface 84. Distal puncture 75 preferably is significantly smaller than proximal puncture 74, for example, twice as small to an order of magnitude or more smaller in cross-sectional diameter.

Referring to FIG. 3C, with distal end 48 of delivery sheath 42 positioned at a desired distance distal of distal puncture 75, the proximal end of push rod 44 is advanced distally by a physician to cause the distal end of push rod 44 to abut the proximal end of bar 22. Push rod 44 is advanced distally until bar 22 is disposed distal of opening 47. Delivery sheath 42 and push rod 44 then may be retracted proximally and removed from the patient's body.

After bar 22 is ejected from delivery sheath 42, filament 26 may be pulled slightly proximally to cause bar 22 to assume an orientation that is substantially parallel to a longitudinal axis of vessel V, as shown in FIG. 3D. When bar 22″ of FIG. 1C is used in place of bar 22, central bore 25 and end eyelet 29 may be utilized to assist in urging bar 22″ to an orientation that is substantially parallel to the vessel when either or both ends of filament 26″ are retracted. In FIG. 3D, first and second ends 27 and 28 of filament 26 are retracted further proximally to retract bar 22 toward distal lateral surface 85 of vessel V.

Referring now to FIG. 3E, tensioning device 90 preferably is used in conjunction with apparatus 40 of the present invention. Tensioning device 90 is similar in structure to a tensioning device previously commercially marketed under the trade name “BioDISC” by BioInterventional Corp. of Pleasanton, Calif. Tensioning device 90 comprises upright 91 having legs 92 and grip 94. Grip 94 may comprise a V-shaped groove in an elastomeric block and retains tension on filament 26 when ends 27 and 28 are pulled distally.

Legs 92 of tensioning device 90 are placed atop an exterior surface of skin S so that space 93, formed between legs 92, is positioned substantially over skin puncture 76. First and second ends 27 and 28 of filament 26 are engaged in grip 94, and may be retracted proximally while legs 92 maintain contact with skin S, as shown in FIG. 3E. The retraction of filament 26 causes bar 22 to provide an internal compressive force upon distal lateral surface 85 of vessel V, while legs 92 provide an external compressive force upon proximal lateral surface 84 through tissue T during tensioning of filament 26.

When the desired tension is provided in filament 26, grip 94 retains ends 27 and 28 in a tensioned state. Advantageously, this permits the internal and external compressive forces described hereinabove to be applied to vessel V without requiring a physician to manually hold ends 27 and 28 of filament 26 for an extended period of time.

The compressive forces imposed upon vessel V cause lumen 72 to narrow locally, which in turn causes coagulation of blood in the vicinity of punctures 74 and 75. Over a period of time, the coagulation occurring within punctures 74 and 75 results in a reduction in diameter of the punctures and halts blood loss from the vessel. Advantageously, the use of bar 22 in conjunction with tensioning device 90 allows compressive forces to be applied to vessel V from substantially diametrically opposing surfaces of vessel V to facilitate closure of punctures 74 and 75. If desired, filament ends 27 and 28 may be intermittently disengaged from grip 94 throughout the procedure to relieve the tensile force imposed upon filament 26 to temporarily reduce the compressive forces applied to vessel V.

If desired, collagen or a biocompatible gel or polymer, such as a water swellable gel or a biodegradable polymer like Polyethylene Glycol (“PEG”), may be injected into puncture tract 70 to reduce a diameter of the puncture tract either before, during or after the time in which the internal compressive force is applied to distal lateral surface 85. If the use of collagen, gels or polymers is employed, it is preferred that the agent is injected into puncture tract 70 while filament 26 is tensioned with tensioning device 90.

Referring now to FIG. 3F, the compressive forces applied to vessel V have caused a significant reduction in the diameter of punctures 74 and 75 to effectively seal the punctures. Filament 26 then may be released from grip 94 to relieve the tensile forces imposed upon filament 26. A physician may proximally retract first end 27 of filament 26 to cause second end 28 to be pulled in a distal direction through skin puncture 76, proximal and distal punctures 74 and 75, and through bore 24 of bar 22. First end 27 further is retracted proximally to remove second end 28 from the patient's body, thereby leaving bar 22 disposed within tissue T. If bar 22 is manufactured from a biocompatible material such as polyglycolic acid, it will be resorbed by the patient's body.

In an alternative approach for sealing puncture 74 of FIG. 3, sharpened tip 45 of sheath 42 may initially pierce substantially deeper into tissue T, i.e., to a location substantially distal of distal lateral surface 85 of vessel V. Accordingly, when bar 22 is ejected from sheath 42, bar 22 is disposed at a location within tissue T that is not in close proximity to distal lateral surface 85. Using this approach, when filament 26 is placed in tension, tissue T, as opposed to bar 22 directly, applies the internal compressive force upon distal lateral surface 85. This approach advantageously may reduce trauma to distal lateral surface 85 of vessel V when compressive forces are imposed as described hereinabove.

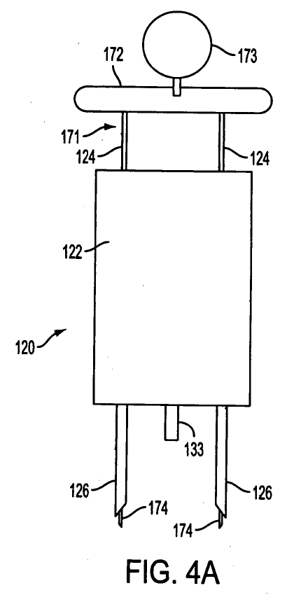

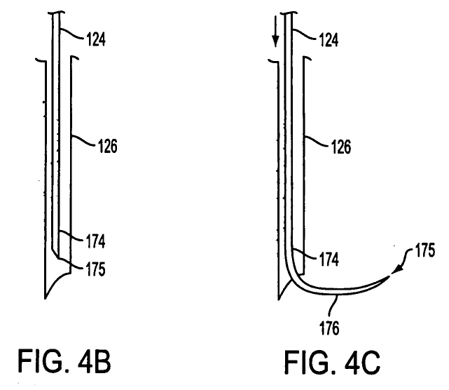

Referring now to FIG. 4, alternative apparatus for sealing punctures in vessels are described. In FIG. 4A, puncture sealing device 120 comprises body 122 having proximal and distal ends, and a portion which is configured to be grasped by a physician. Body 122 includes hollow needles 126, which are configured to pierce through a patient's tissue.

Puncture sealing device 120 further comprises wires 124 having proximal ends 171 that may be translated by advancing or retracting actuator 172 using ring 173. Wires 124 include distal ends 174 having sharpened tips 175, and are sized to be translated through hollow needles 126. Wires 124 have a contracted state in which distal ends 174 are confined within hollow needles 126, as shown in FIG. 4B. Wires 124 are configured to self-deploy to a predetermined shape in a deployed state when distal ends 174 are no longer constrained within hollow needles 126, whereby distal ends 174 curve to form, for example, hook 176, as shown in FIG. 4C.

Wires 124 preferably are manufactured from a shape-memory material, such as a nickel-titanium alloy, that allows distal ends 175 to deploy to the desired shape when no longer constrained within hollow needles 126. The desired deployment shape may be set by applying an appropriate heat treatment to wires 124, which is per se known in the art.

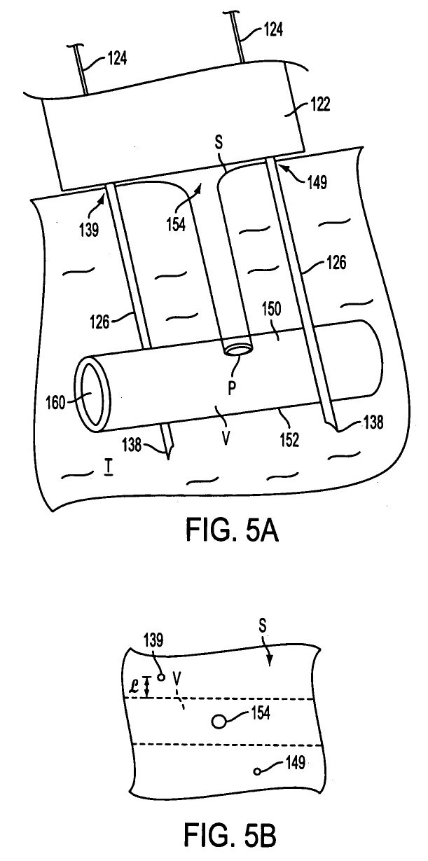

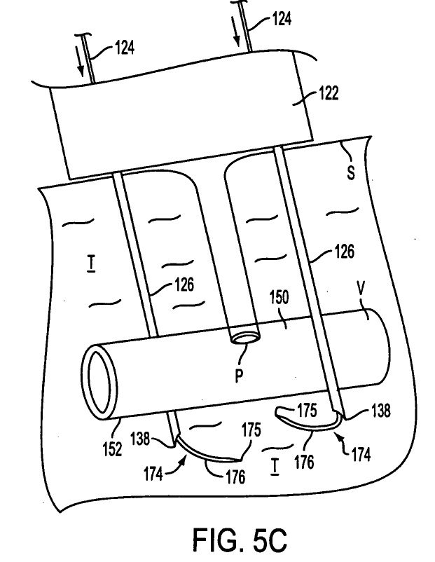

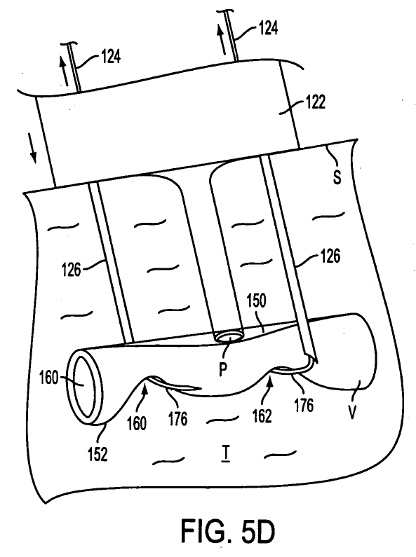

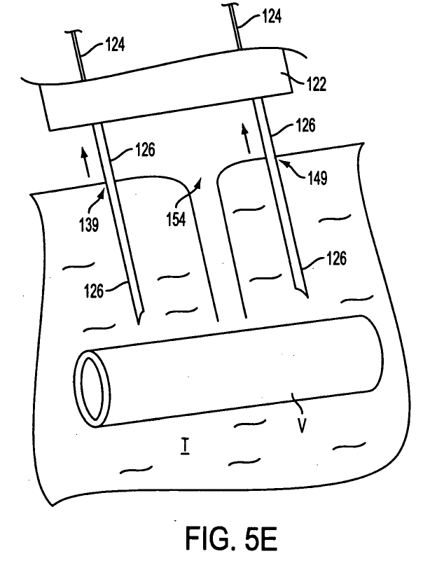

Referring now to FIG. 5, an exemplary method of using the puncture sealing device 120 of FIG. 4 to seal a vessel puncture is described. In FIG. 5A, puncture P has been formed in proximal lateral surface 150 of vessel V, which is situated within tissue T. Puncture tract 154 has been formed in an exterior surface of skin S and is in fluid communication with puncture P of vessel V.

In a first step, hollow needles 126 of device 120 pierce skin S at locations 139 and 149. Locations 139 and 149 preferably are disposed on opposing lateral sides of vessel V, as shown from a top view in FIG. 5B, and are a lateral distance Y from an exterior surface of vessel V to ensure that hollow needles 126 do not pierce through the vessel wall. Body 122 of puncture sealing device 120 optionally may comprise centering shaft 133, as shown in FIG. 4A, which is adapted for placement within puncture tract 154 and/or puncture P to facilitate proper positioning of hollow needles 126 at locations 139 and 149. It is desirable that hollow needles 126 comprise an external diameter that is smaller than the diameter of, for example, puncture tract 154, as shown in FIG. 5B.

Referring back to FIG. 5A, hollow needles 126 pierce through tissue T surrounding vessel V and are advanced distally so that tips 138 of needles 126 are positioned distal of distal lateral surface 152 of vessel V. Wires 124 are provided in contracted states within hollow needles 126. After hollow needles 126 are positioned at the desired distance distal of distal lateral surface 152, wires 124 are advanced distally with respect to hollow needles 126 such that distal ends 174 of wires 124 are no longer constrained within needles 126. Distal ends 174 self-deploy to their predetermined shapes, e.g., hook shapes 176. Sharpened tips 175 at the distal ends of wires 124 pierce through tissue T as the wires self-deploy to form hooks 176, as shown in FIG. 5C, preferably with wires 124 forming substantially opposing hooks 176.

Referring now to FIG. 5D, ring 173 may be actuated to retract actuator 172 and wires 124 proximally to cause hooks 176 to apply an internal compressive force upon distal lateral surface 152 of vessel V at first region 160 and second region 162. Preferably, needles 126 are proximally retracted with wires 124 such that surface friction applied by tissue T to hooks 176 causes the hooks to apply the internal compressive force, as opposed to retracting within the needles.

Hooks 176 may either engage vessel V, as shown in FIG. 5D, or alternatively may remain engaged with tissue T distal of distal lateral surface 152, so that tissue T effectively applies the compressive force upon distal lateral surface 152. In addition, an external compressive force may be applied by the distal end of body 122. Body 122 may be configured for longitudinal movement with respect to wires 124 and needles 126 so that body 122 may be urged distally against skin S while needles 126 and wires 124 are retracted proximally. Additionally, collagen, polymers and/or gels may be disposed within puncture tract 154, as described hereinabove with respect to FIG. 3E.

The compressive forces provided by hooks 176 and body 122 may be applied for a period of several minutes to disrupt blood flow in lumen 160 of vessel V. Specifically, the disruption in local blood flow causes coagulation in puncture P that facilitates closure of the puncture.

After compression has been applied for a desired period of time via hooks 176 and, optionally, body 122 to seal puncture P, the compressive force applied by hooks 176 upon distal lateral surface 152 may be relieved by distally advancing hollow needles 126 while holding body 122 stationary. Alternatively, ring 173 may be retracted proximally while needles 126 are held stationary, thereby applying a contact force between hooks 176 and needles 126 that is expected to overcome surface friction applied by tissue T to hooks 176. Distal ends 175 of wires 124 collapse within hollow needles 126.

At this time, hollow needles 126 and wires 124 then may be retracted proximally simultaneously and removed through punctures 139 and 149, as shown in FIG. 5E. Bandages and/or sutures then may be applied to skin punctures 139, 149 and 154 to promote healing of the skin punctures upon completion of the procedure.

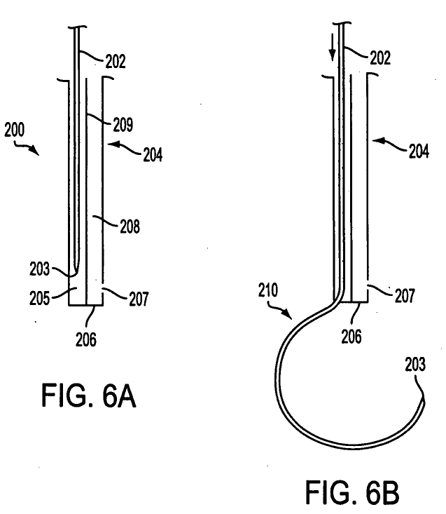

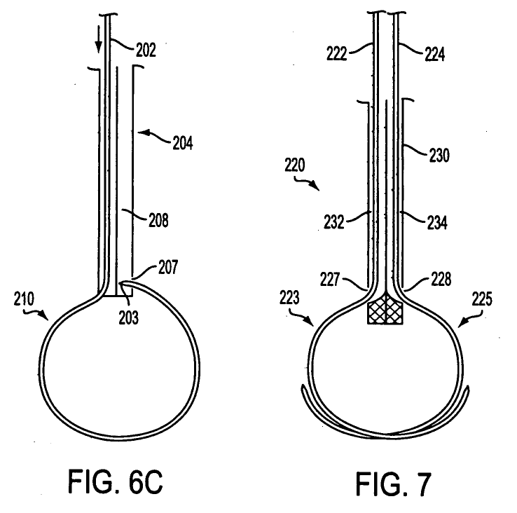

Referring now to FIGS. 6-7, yet further alternative embodiments of the present invention are described. In FIG. 6A, puncture sealing device 200 comprises at least one wire 202 configured to self-deploy to a predetermined shape. Wire 202 preferably comprises a shape-memory material and includes a distal end having sharpened tip 203, which is configured to pierce through tissue surrounding the vessel, and a proximal end (not shown) that may be manipulated by a physician. Wire 202 is provided in a contracted state within delivery sheath 204 having proximal and distal ends. Delivery sheath 204 preferably comprises atraumatic tip 206 disposed at the distal end, but optionally may comprise a sharpened tip or another configuration.

Preferably, delivery sheath 204 comprises a first lumen 205 having a diameter that is slightly larger than the diameter of wire 202 to constrain wire 202 in the contracted state. Partition 209 may be used to divide delivery sheath 204 into a plurality of lumens. Alternatively, sheath 204 may be dimensioned such that it has only first lumen 205 with wire 202 disposed concentrically therein. When wire 202 is advanced distal of delivery sheath 204, a distal segment of wire 202 is configured to self-deploy to a predetermined arcuate shape, for example, at least a semicircular shape, as shown in FIG. 6B, or a fully circular shape, as shown in FIG. 6C. In these deployed states, the radius of curvature of arcuate hook 210 preferably is greater than a radius of curvature of the vessel, so that hook 210 may readily surround the vessel while reducing a risk of piercing a lateral surface of the vessel.

The distal end of wire 202 may be advanced distally through an opening at a distalmost end of delivery sheath 204, as illustratively shown in FIG. 6B, or alternatively may exit through a side port, for example, as illustratively shown by side ports 227 and 228 of the embodiment of FIG. 7. Delivery sheath 204 further may be provided with wire capture element 207, which is illustratively shown in FIG. 6C as a side port disposed in a lateral surface of delivery sheath 204. Capture element 207 is configured to engage the distal end of wire 202 when wire 202 deploys to a fully circular shape. It will be apparent to those skilled in the art that a locking mechanism (not shown) alternatively may be provided on delivery sheath 204 or disposed within second lumen 208 to engage the distal end of wire 202. Alternatively, secondary apparatus (not shown) may be provided to capture wire 202.

Referring now to FIG. 7, first and second wires 222 and 224 are provided within first and second lumens 232 and 234 of delivery sheath 230, respectively. Wires 222 and 224 are provided in contracted states whereby distal ends of each wire are constrained within the confines of delivery sheath 230, e.g., as described with respect to the embodiment of FIG. 6A.

Wires 222 and 224 are advanced distally, either individually or simultaneously, so that the distal end of wire 222 exits through side port 227 and the distal end of wire 224 exits through side port 228. As the wires further are advanced distally, they assume their respective predetermined arcuate shapes, whereby the distal end of wire 222 forms arcuate hook 223 and the distal end of wire 224 forms arcuate hook 225. Arcuate hooks 223 and 225 preferably deploy in opposing directions such that each wire initially bows outwardly from delivery sheath 230, then curves back towards sheath 230 and eventually crosses paths with the opposing wire, as shown in FIG. 7.

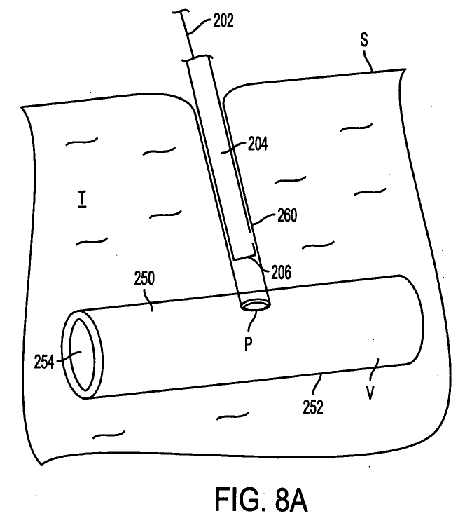

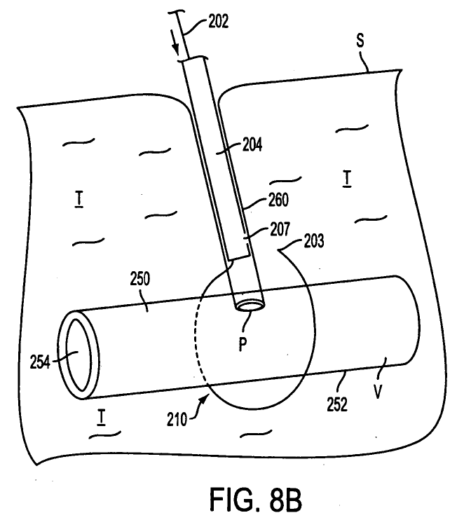

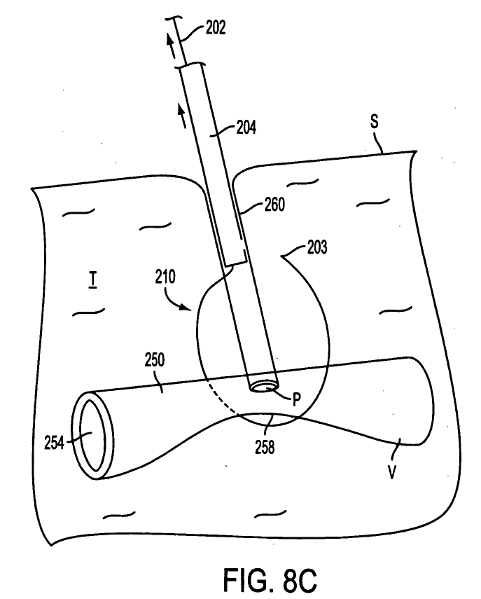

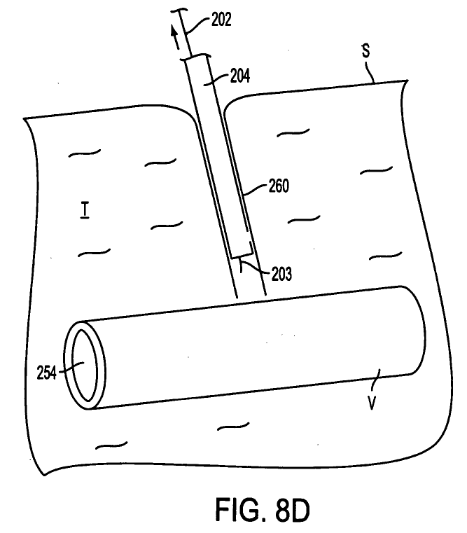

Referring now to FIGS. BA-8D, an exemplary method of using apparatus 200 of FIGS. 6A-6C is described. It will be apparent to those skilled in the art that the method described in FIGS. 8A-8D also may be suitable for using device 2120 of FIG. 7. In operation, delivery sheath 204 is placed in puncture tract 260 leading to vascular puncture P, as shown in FIG. 8A. Sheath 204 optionally may be advanced into position within the puncture tract through, for example, an introducer or a guide catheter, which are per se known in the art. Additionally, the distal end of sheath 204 optionally may be advanced through puncture P into lumen 254 of vessel V, such that a pressure differential between atmospheric pressure and blood pressure within the vessel causes blood to flow through lumen 205 of sheath 204, and out a proximal end of the sheath, thereby providing backbleed indication of proper positioning. When using the distal end of delivery sheath 204 for backbleed indication, wires disposed within sheath 204 preferably exit through side ports, as opposed to the distal end of the sheath, such that the wires do not enter the lumen of the vessel. As will be apparent to those skilled in the art, a dedicated second lumen or tube (not shown) may be provided with sheath 204 for backbleed indication.

At this time, one or more wires 202 are provided in the contracted state within the confines of delivery sheath 204, e.g., as shown in FIG. 6A. Atraumatic tip 206 of delivery sheath 204 preferably is positioned within puncture tract 260 and is configured such that one or more wires 202 may exit delivery sheath 204 in proximity to vessel V without entering lumen 254 of vessel V. For example, delivery sheath 204 may be positioned in puncture tract 260 such that wire 202 may exit a distal end of sheath 204 and be positioned in the puncture tract proximal of puncture P. Alternatively, sheath 204 may comprise one or more side ports, e.g., side ports 227 and 228 of FIG. 7, through which the one or more wires may exit the sheath proximal of puncture P.

As wire 202 exits sheath 204, wire 202 assumes its predetermined, preferably arcuate shape. As shown in FIG. 8B, wire 202 circumferentially pierces through tissue T surrounding vessel V until the distal end of wire 202 has at least crossed an imaginary plane on distal side 252 of vessel V, the imaginary plane including a diameter of the vessel passing from puncture P through the vessel's midpoint to distal side 252 of vessel V. As described hereinabove, wire 202 preferably assumes at least a semi-circular shape, as shown in FIG. 6B, or a fully circular shape, as shown in FIG. 6C. When a fully circular shape is formed, the distal end of wire 202 may engage capture element 207 of sheath 204.

Once positioned in the deployed state across the imaginary plane, wire 202 and, preferably, sheath 204 may be retracted proximally simultaneously to cause arcuate hook 210 of wire 202 to apply an internal compressive force upon vessel V near vessel region 258, as shown in FIG. 8C. It is expected that surface friction along the length of wire 202 disposed within tissue T will cause wire 202 to apply the internal compressive force, as opposed to retracting from tissue T. The internal compressive force applied by wire 202 causes coagulation in the vicinity of puncture P that facilitates sealing of the puncture. To expedite sealing of the puncture, a proximal end of the one or more wires may be tensioned within previously described tensioning device 90 of FIGS. 3E-3F, which may be disposed on an exterior surface of a patient's skin S. The proximal end of wire 202 may be locked within tensioning device 90 so that the physician need not manually retain the compression upon the vessel. Additionally, the tensioning device serves to apply an external compressive force to the skin to enhance compression of the vessel.

After an appropriate period of time has elapsed, tension upon sheath 204 and/or wire 202 are relieved. Wire 202 then may be contracted by proximally retracting wire 202 with respect to sheath 204 to cause arcuate hook 210 to be contracted within the distal end of sheath 204, as shown in FIG. 8D. It is expected that a contact force applied at the point where wire 202 exits and contacts sheath 204 will overcome the surface friction applied to the length of wire 202 disposed within tissue T, thereby facilitating removal of wire 202 from the tissue into the sheath. Once the distal end of wire 202 is disposed within the confines of sheath 204, sheath 204 and wire 202 may be retracted simultaneously and removed from puncture tract 260.

When using device 220 of FIG. 7 in accordance with method steps described in FIGS. BA-8D, wires 222-and 224 may cross the imaginary plane from opposite sides such that they overlap distal of vessel V. Advantageously, the compressive forces applied to vessel V are distributed between the two wires, thereby reducing a risk of vessel perforation or dissection. It will be apparent to those skilled in the art that any number of wires having predetermined shapes may be provided to achieve the functions described hereinabove.

Moreover, it will be apparent to those skilled in the art that the figures accompanying the preferred embodiments are provided only for the sake of illustration and are not drawn to scale. For example, it is expected that the diameter of needles 126 of FIGS. 4-5 and the diameter of distal end 48 of delivery sheath 42 of FIGS. 3B-3C, as well as the diameters of all apparatus disposed therein, will be significantly smaller than the diameter of puncture P or vessel V. Additionally, in FIG. 3, the diameter of distal puncture 75 is expected to be significantly smaller than the diameter of proximal puncture 74.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

1. Apparatus for facilitating sealing of a puncture formed in a proximal lateral surface of a vessel, the apparatus comprising:
a bar having proximal and distal ends and a first bore extending laterally therethrough; and
a filament disposed through the first bore,
wherein the bar is configured to apply a compressive force upon a distal lateral surface of a vessel.

2. The apparatus of claim 1 further comprising a delivery sheath having proximal and distal ends, a lumen extending therebetween and a sharpened tip at the distal end, wherein the lumen is configured to contain the bar and filament.

3. The apparatus of claim 2 further comprising a push rod disposed in the lumen proximal of the bar.

4. The apparatus of claim 1, 2 or 3 wherein the bar has a shape chosen from the group consisting of cylindrical shapes, rectangular shapes, and oval shapes.

5. The apparatus of claim 1, 2, 3 or 4 wherein the bar comprises a biodegradable material.

6. The apparatus of claim 1, 2, 3, 4 or 5 further comprising a tensioning device configured to hold the filament in a tensioned state.

7. The apparatus of claim 6 wherein the tensioning device comprises:
an upright having upper and lower ends;
a plurality of legs attached to the lower end; and
a grip affixed to the upper end.

8. The apparatus of claim 7 wherein the grip comprises a V-shaped groove formed in an elastomeric material.

9. The apparatus of any one of claims 1 to 8 wherein the first bore is disposed in a central region of the bar.

10. The apparatus of any one of claims 1 to 9 further comprising an eyelet coupled to the bar, wherein the filament is disposed through both the first bore and the eyelet.

11. The apparatus of claim 10 wherein the first bore is disposed in a central region of the bar, and the eyelet is coupled to a distal region of the bar.

12. The apparatus of claim 10 or 11 further comprising an eyelet coupled to the bar, wherein the eyelet is coupled to a central region of the bar, and the first bore is disposed in a distal region of the bar.

13. The apparatus of any one of claims 1 to 12 further comprising a second bore extending laterally through the bar, wherein the filament is disposed through both the first bore and the second bore.

14. The apparatus of claim 13 whrerein the first bore is disposed in a central region of the bar, and the second bore is disposed in a distal region of the bar.

15. Apparatus for facilitating sealing of a puncture formed in a proximal lateral surface of a vessel, the apparatus comprising:
a bar having proximal and distal ends and a first eyelet coupled to the bar; and
a filament disposed through the first eyelet,
wherein the bar is configured to apply a compressive force upon a distal lateral surface of a vessel.

16. The apparatus of claim 15 wherein the first eyelet is coupled to a central region of the bar.

17. The apparatus of claim 15 or 16 wherein the bar further comprises a second eyelet coupled to the bar, wherein the filament is disposed through the first eyelet and the second eyelet.

18. The apparatus of claim 17 wherein the first eyelet is coupled to a central region of the bar, and the second eyelet is coupled to a distal region of the bar.

19. The apparatus of claim 15, 16, 17 or 18 further comprising a delivery sheath having proximal and distal ends, a lumen extending therebetween and a sharpened tip at the distal end, wherein the lumen is configured to contain the bar and filament.

20. The apparatus of claim 19 further comprising a push rod disposed in the lumen of the delivery sheath proximal of the bar.

21. The apparatus of any one of claims 15 to 20 wherein the bar comprises a biodegradable material.

22. The apparatus of any one of claims 15 to 21 further comprising a tensioning device configured to hold the filament in a tensioned state.

23. Apparatus for facilitating sealing of a puncture formed in a proximal lateral surface of a vessel, the apparatus comprising:
a body having proximal and distal ends and a first tube extending from the distal end of the body; and
a first wire having proximal and distal ends and a sharpened tip at the distal end, wherein the distal end of the first wire is configured for constraint within the first tube in a contracted state, and further configured to self-deploy to a predetermined shape in a deployed state when the distal end is no longer constrained within the first tube,
wherein the first wire is configured to engage tissue distal of a distal lateral surface of the vessel to apply a compressive force thereto.

24. The apparatus of claim 23 wherein the first wire comprises a shape-memory material.

25. The apparatus of claim 23 or 24 wherein the first tube comprises a first needle, the apparatus further comprising:
a second needle extending from the distal end of the body; and
a second wire having proximal and distal ends and a sharpened tip at the distal end, the second wire having a contracted state configured for constraint within the second needle, and a deployed state.

26. The apparatus of claim 25 wherein the sharpened tip of the first wire is configured to deploy in a direction opposing a direction of the sharpened tip of the second wire when deployed.

27. The apparatus of claim 25 or 26 wherein the first needle is adapted to pierce tissue on a first lateral side of the vessel and the second needle is adapted to pierce tissue on a second lateral side of the vessel.

28. The apparatus of claim 27 wherein the body further comprises a centering shaft adapted for placement within a puncture tract extending through tissue to the puncture, thereby facilitating proper positioning of the first and second needles on the first and second lateral sides of the vessel, respectively.

29. Apparatus for facilitating sealing of a puncture formed in a proximal lateral surface of a vessel, the apparatus comprising:
a delivery sheath having proximal and distal ends and a first lumen extending therebetween; and
a first wire having proximal and distal ends and a sharpened tip at the distal end, wherein the distal end of the first wire is configured for constraint within the first lumen of the delivery sheath in a contracted state, and further configured to self-deploy to a predetermined shape in a deployed state when the distal end is no longer constrained within the delivery sheath,
wherein the first wire is configured to engage tissue distal of a distal lateral surface of the vessel to apply a compressive force thereto.

30. The apparatus of claim 29 wherein the predetermined shape of the first wire comprises a shape chosen from the group consisting of hook shapes, arcuate shapes, arcuate shapes having a radius of curvature larger than a radius of curvature of the vessel, semi-circular shapes, and circular shapes.

31. The apparatus of claim 29 or 30 wherein the delivery sheath is adapted for placement within a puncture tract, wherein the puncture tract extends through tissue to the puncture.

32. The apparatus of claim 29, 30 or 31 wherein the delivery sheath further comprises at least one side port through which the distal end of the first wire may be advanced.

33. The apparatus of claim 29, 30, 31 or 32 further comprising a second wire having proximal and distal ends and a sharpened tip at the distal end, the second wire having a contracted state whereby the second wire is constrained within a second lumen of the delivery sheath, and a deployed state.

34. The apparatus of claim 33 wherein the sharpened tip of the first wire is configured to deploy in a direction opposing a deployed direction of the sharpened tip of the second wire.

35. The apparatus of any one of claims 29 to 34 further comprising a tensioning device configured to hold the first wire in a tensioned state.
